(19) United States
(12) Patent Application Publication
(10) Pub. No.: US 2003/0017448 A1
(43) Pub. Date: Jan. 23, 2003

(54) Method of prophylaxis and treatment of porcine reproductive and respiratory syndrome

(75) Inventor: Fernando A. Osorio, Lincoln, NE (US)

Correspondence Address: SENNIGER POWERS LEAVITT AND ROEDEL, ONE METROPOLITAN SQUARE, 16TH FLOOR, ST LOUIS, MO, 63102, US

(73) Assignee: The Board of Regents of the University of Nebraska

(21) Appl. No.: 10/081,753

(22) Filed: Feb. 22, 2002

Related U.S. Application Data
(60) Provisional application No. 60/271,284, filed on Feb. 23, 2001.

(51) Int. Cl. C12Q 1/70; C07H 21/04; C12P 19/34; A61K 39/42; C12N 5/06; C12N 5/16

(52) U.S. Cl. 435/5; 435/345; 435/91.1; 536/23.72; 424/147.1

(57) ABSTRACT

A method for the prevention of intra uterine infection of fetuses with porcine reproductive and respiratory syndrome virus (PRRSV) is provided comprising administering to a pregnant female a composition containing an effective dose of anti-PRRSV antibodies. Also provided is a method for the prophylaxis or treatment of porcine reproductive and respiratory syndrome comprising administering to a pig a composition comprising an effective amount of anti-PRRS virus antibodies.

### CROSS-REFERENCE TO RELATED APPLICATION

This application claims priority from Provisional Application Serial No. 60/271,284 filed on Feb. 23, 2001, which is hereby incorporated by reference in its entirety.

### STATEMENT REGARDING FEDERALLY FUNDED RESEARCH

This invention was made, in part, with government support under grant number NRICGP/USDA 99-35204-8041 awarded by the United States Department of Agriculture. The government has certain rights in this invention.

### BACKGROUND

Porcine Reproductive and Respiratory Syndrome (PRRS), also known as Mystery Disease of Swine, was initially described in the USA in 1987 and is now endemic in many swine-producing countries. The causative agent, Porcine Reproductive and Respiratory Syndrome Virus (PRRSV) is a member of a group of enveloped single-stranded RNA viruses classified in the order Nidovirales, family Arteriviridae, and genus Arterivirus. Arteriviruses are spherical, single-stranded, positive sense RNA viruses with similar structural and functional organizations. They are characterized by six to eight subgenomic mRNAs with a common 5′ leader sequence. In PRRSV, proteins for ORFs 5, 6 and 7 are associated with the envelop, virus membrane protein, and nucleocapsid, respectively. These envelop and membrane proteins are thought to be important for virus-host interaction.

The predominant route of transmission of PRRSV is by direct contact between infected and naive pigs. PRRSV has been isolated from, or identified in, semen, saliva, feces, urine, nasal swabs, oropharyngeal swabs, and oropharyngeal scrapings. In utero infection of developing fetuses can occur by transplacental migration of the virus.

Clinical manifestation of PRRS varies with the age, pregnancy status, and trimester of gestation of the infected individual. Infection of pregnant animals in the third trimester of gestation can result in abortions, the incidence of which varies from sporadic to widespread. Not all animals are initially infected so that reproductive failure may persist for up to six months. PRRSV infection during the last trimester of gestation is characterized by late term abortion and the birth of stillborn or mummified fetuses. In addition, surviving fetuses are often infected shortly after birth or in utero due to transplacental migration of the virus and develop clinical symptoms. Neonatal pigs infected with PRRSV usually develop severe dyspnea (thumping) and tachypnea. In addition, infected neonates also exhibit periocular edema, conjunctivitis, eyelid edema, blue discoloration of the ears, fever, cutaneous erythema, diarrhea, rough hair coats, postinjection bleeding, and central nervous system disorders. Mortality in neonatal pigs with PRRS may reach 100%. Pigs that do survive show decreased growth rates and feed efficiency. PRRSV infection in weaned pigs is characterized by fever, pneumonia, lethargy, failure to thrive, and a marked increase in mortality from single to multiple concurrent bacterial infections.

The cost effects of PRRSV infection can be significant. Initial estimates indicated that losses to the swine industry due to PRRSV-induced reproductive failure could amount to $500 per inventoried female in addition to the increase cost associated with decreased growth rate of infected pigs. Other estimates have put the cost of PRRSV infection at $5 to $15 per pig produced. Considering the number of pigs produced annually in the United States alone, it is apparent that PRRSV results in annual losses and increased production cost in the millions of dollars.

Because of the substantial costs involved, considerable research has been conducted regarding diagnosis, treatment and prevention of PRRS. Methods for growing PRRS virus in culture and for producing attenuated virus for use in vaccines can be found, for example, in U.S. Pat. Nos. 5,510,258; 5,840,563; 5,846,805; 5,989,563; and 6,080.570. Many preparations have been developed for the prevention, treatment and diagnosis of PRRS including those found in U.S. Pat. Nos. 5,587,164; 5,888,513; 5,620,691; 5,690.940; 5,698,203; 5,858,729; 5,866,401; 5,976,537; 5,998,601; 6,001,370; 6,042,830; 6,110,468; 6,033,844; 5,683,865; 5,677,429 and European Patent No. EP 0601062. Although vaccines are able to protect pigs from PRRSV infection in many cases, the vaccines have failed in some cases to provide protection against especially virulent forms of the virus. In addition, two manufacturers of commercially available PRRSV vaccines do not recommend that their vaccines be administered during gestation, presumably because attenuated virus and virulent field virus can cross the placenta and infect the fetus (Mengeling et al., Am. J Vet. Res., 59:52-55, 1998; Mengeling et al., Am. J Vet. Res., 60:796-801, 1999). Prevention from infection in utero with PRRSV may be especially important, since at least one report indicates that pigs born infected with PRRSV become persistently infected, providing a source of virus within the herd (Benfield et al., 1998 Allen D. Leman Swine Conf, pp.169-171).

Although it is known that prior infection with PRRSV will lead to immunity, the mechanism by which such immunity is conferred has not been clearly established. Exposure to a virus typically results in the body mounting both a humoral (antibody) and cell mediated response. Depending on the antibody detection method used, anti-PRRSV IgM antibodies can be detected between 7 and 10 days post infection (dpi). Anti-PRRSV serum neutralizing antibodies develop from 9 to 105 dpi (Rossow, Vet. Pathol., 35:1-20, 1998). Many authors, however, have questioned the significance of humoral immunity protection against PRRSV (Loemba et al., Arch. Virol., 141:751-761, 1996). It is known that PRRSV can replicate in and spread from pigs with neutralizing antibodies (Rossow, Id.). This led Rossow to conclude that serum-neutralizing antibodies were not necessarily an essential part of the immune response to PRRSV. Labarque et al. (J Gen. Virol. 81:1327-1334, 2000) reported that low amounts of PRRSV remained in the lungs of infected animals in spite of the presence of neutralizing antibodies leading the authors to conclude that cell mediated immunity was involved in the removal of the virus from the lungs. In addition, the observation that antibodies could enhance PRRSV replication in macrophages (Yoon et al., Viral Immunol., 9:51-63, 1996) is considered additional .evidence that PRRSV antibodies constitute a deleterious, non-protective response.

There are reports that ingestion of colostrum can provide protection against PRRSV infection (Morrison et al., Proc. 14^{th} IPVS Cong., Bologna, Italy, 1996, p 60; Gorcyca et al., Proc. 14^{th} IPVS Cong., Bologna, Italy, 1996, p. 61). These results do not, however, provide unequivocal evidence that the passive transfer of anti PRRSV antibodies will provide protection since colostrum contains both antibodies and leukocytes. To address this question, Molitor (Proc. Allen D Leman Swine Conf., 20: 49-50, 1993) reported that passive transfer of antibodies from colostrum did not provide protection against PRRSV infection. This led the author along with others in the field to conclude that passive immunization with anti PRRSV antibodies was insufficient to protect pigs against subsequent challenge with the virus (Bautista et al., Proc. 14^{th} IPVS Cong., Bologna, Italy, 1996; Collins, Proc. 15^{th} IPVS Cong. pp. 149-156, 1998; Molitor et al., Vet Microbiol. 55:265-276, 1997).

A need therefore, exists for a safe, effective means of protecting fetuses from in utero infection with PRRSV. Ideally, this method should not rely on live virus in order to reduce the risk of infection from vaccination. Contrary to the prevailing opinion in the field, the present inventor has discovered that passive immunization of pregnant females with anti PRRSV antibodies prevents in utero infection of the fetuses with PRRSV upon subsequent challenge. Thus, the present invention provides a safe and effective method for the prevention of fetal infection by PRRSV. The present invention can also be used as a means for the treatment or prophylaxis of PRRSV.

### SUMMARY

Accordingly, among the several aspects of the invention is provided, a method for the prevention of intra uterine infection of fetuses with Porcine Reproductive and Respiratory Syndrome Virus (PRRSV) comprising, administering to a pregnant female a composition containing effective dose of anti-PRRSV antibodies. In one aspect, the composition is administered during the last trimester of gestation.

In another aspect a method for the prevention of intra uterine infection of fetuses with Porcine Reproductive and Respiratory Syndrome Virus (PRRSV) is provided comprising parenterally administering to a pregnant female during the last trimester of gestation an effective amount of a composition containing anti-PRRSV antibodies.

Yet another aspect provides a method for the prophylaxis or treatment of Porcine Reproductive and Respiratory Syndrome (PRRS) in swine comprising administering to a pig a composition comprising an effective amount of anti-PRRS virus antibodies. In one aspect the composition is administered during the last trimester of pregnancy.

Still another aspect provides a method for the prophylaxis or treatment of Porcine Reproductive and Respiratory Syndrome (PRRS) in swine comprising parenterally administering to a pig a composition comprising an effective amount of anti-PRRS virus antibodies.

In all of the above aspects a composition is provided which comprises polyclonal antibodies, monoclonal antibodies, recombinant antibodies, or combinations thereof. Further provided are therapeutic combinations which also include vaccines against diseases which porcines are susceptible.

Other objects and features will be in part apparent and in part pointed out hereinafter.

### BRIEF DESCRIPTION OF THE DRAWINGS

These and other features, aspects, and advantages of the present invention will become better understood with regard to the following description, appended claims and accompanying figures where:

FIG. 1 shows the design of the passive transfer experiment to test the role of neutralizing antibodies in in vivo protection against Porcine Reproductive and Respiratory Syndrome Virus induced reproductive failure.

FIG. 2 shows the Porcine Reproductive and Respiratory Syndrome Virus serological profiles in the principal and Ab-specificity control groups: mean (n=6) of ELISA (S/P) and SN titers. Solid arrow indicates means date of farrowing.

### ABBREVIATIONS

ELISA=enzyme-linked immunosorbent assay

S/P=Signal-to-Positive ratio

SN=serum neutralizing

wt=wild type

ORF=open reading frame

Ab=antibody

Ig=immunoglobulin

IFN=interferon

IP=intraperitoneally

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, databases and other references cited in this application are herein incorporated by reference in their entirety as if each individual publication, patent, patent application, database or other reference were specifically and individually indicated to be incorporated by reference.

The present inventor has surprisingly found that intra uterine infection of fetuses with the Porcine Reproductive and Respiratory Virus (PRRSV) can be prevented by the administration to a female of an effective amount of a composition comprising anti-PRRSV antibodies. In one embodiment, the antibody containing composition is administered during the last trimester of pregnancy. The present discovery also has application for the prophylaxis or treatment of PRRS. In this embodiment, an animal infected with PRRSV or susceptible to PRRSV infection is administered a composition containing an effective amount of PRRSV antibodies. The composition can be administered at any time during the lifetime of the animal. For example, the antibody composition can be administered to a naive animal immediately prior to its introduction into a herd known or suspected to contain infected animals. Alternatively, the antibody containing composition can be administered to newborn animals to increase protection against infection with PRRSV. In all of the preceding embodiments, it is preferred that the antibody containing composition be administered prior to exposure to the PRRS virus, however, administration of the composition after exposure is also contemplated and within the scope of the present invention.

The antibody containing composition can be administered alone or in conjunction with a vaccine. For example and without limitation, prior to the introduction of a naive animal into a herd or environment known or suspected to be infected with the PRRS virus, the animal can be administered the antibody containing composition in accordance with the present invention to provide immediate protection and a vaccine to provide active immunization against the PRRS virus. In order to prevent interference between the antibodies in the composition administered with the antigenic determinates of the vaccine, it is preferred that the antibodies administered be directed to different epitopes or viral strains than those contained in the vaccine. In addition to vaccines directed against the PRRS virus, vaccines directed against other diseases and/or organisms can also be administered in conjunction with the antibody containing composition. Examples of vaccines that can be administered include, but are not limited to, hog cholera, transmissible gastroenteritis, swine influenza, porcine parvovirus, swine enzootic pneumonia, pseudorabies, encephalomyocarditis, Japanese encephalitis, rotavirus, leptospirosis, erysipelas, brucellosis, and enterovirus, as well as any combination of the preceding.

The vaccine can comprise live virus, either unaltered or attenuated, or inactivated virus. The vaccine can alternatively contain viral proteins or peptides, for example viral coat proteins or peptides. In another embodiment, the vaccine is a DNA vaccine. By “in conjunction with” is meant that the vaccine is administered within a short time, preferably within ± one week of the administration of the anti-PRRSV antibody containing composition of the present invention. When the vaccine is administered at the same time as the anti-PRRSV antibody containing composition the vaccine can be incorporated into the antibody containing composition or can be administered as a separate composition.

The antibodies administered can be obtained from a variety of sources including polyclonal antibodies, monoclonal antibodies, and recombinant antibodies. The antibodies used can be from the same species or obtained from a different species as the animal to which the antibodies are to be administered. The antibodies can also be against the same strain of PRRSV against which protection or treatment is sought or can be against a different strain. In one embodiment, the antibodies used are from the same species as the animal to which they are administered, i.e. porcine, and are against the same strain of the PRRS virus against which protection is sought. Methods for the production of polyclonal antibodies and antisera are well known in the art and can be found in standard references such as Ausubel et al., Short Protocols in Molecular Biology, 3^{rd} ed, Wiley Publishing, 1995, unit 11.5. Briefly, an immunocompetent animal is exposed to an antigen against which antibodies are to be obtained. The antigen can take many forms. The antigen can be a whole organism, for example, the PRRS virus or the antigen can be a molecule, typically a protein, glycoprotein, or carbohydrate moiety obtained from the organism. When the whole organism is used, the native, that is unmodified, form of the organism can be used or an attenuated form can be used. Attenuation is typically achieved by multiple passages of the organism through animals or cell lines of a different species than that which is normally infected by the organism. For example, U.S. Pat. No. 6,080,570 discloses the use of passage through a simian cell line as a means of attenuation, however, any cell line or animal that will allow growth of the virus can be used. Attenuation can also be achieved by the use of recombinant DNA technology. In this method of attenuation, the organism is genetically modified using molecular biology techniques to decrease its virulence while maintaining its antigenic properties. For example, genes associated with the growth or some aspect of the organism associated with pathogenicity can be mutated or deleted. Methods for the genetic modification of prokaryotes and in particular viruses are well known in the art. Alternatively, an inactivated, e.g. killed, organism can be used. Any suitable method of inactivation can be used including, but not limited to heat inactivation and chemical inactivation.

Polyclonal antibodies or antisera can also be produced using proteins, glycoproteins, peptides or fragments thereof. Antigenic proteins can be extracted and, if desired, purified from the organism of interest using standard biochemical techniques. More typically, proteins or peptides are produced using standard recombinant DNA technology methods. Briefly, nucleotide sequences encoding the proteins or peptides of interest are isolated and inserted into expression vectors, which in turn are introduced into suitable host cells. These host cells are then grown under conditions appropriate for expression of the protein(s) of interest. The proteins can then be obtained by disruption of the cells, or, if the proteins are secreted, from the culture medium. Preferably, the proteins of interest are then purified using standard techniques. In the case of PRRSV, proteins encoded by open reading frames (ORF) 2-7 are typically used. If desired, either naturally occurring or recombinant proteins can be chemically coupled to carrier proteins or molecules as described for example, in Ausubel et al., unit 11.8. The recombinant proteins produced can comprise only the protein of interest or can comprise fusion proteins. Fusion proteins or chimeric proteins can contain additional amino acid sequences useful to increase the antigenicity of the protein produced, to aid in the purification of the recombinant protein, or both. Production of fusion proteins is well known in the art and can readily be accomplished by the skilled technician using standard techniques such as those described in Ausubel et al., and Sambrook et al, Molecular Cloning, 2^{nd} ed., Cold Spring Harbor Laboratory Press, 1989.

Once obtained, the antigen is administered to the animal to elicit an immune response. Any suitable means of administration can be used. Typically the antigen is administered parenterally. Examples of suitable means of parenteral administration include intramuscular injection, subcutaneous injection, intradermal injection, intravenous injection, and intraperitoneal injection. Additionally, the antigen can be administered via the respiratory tract, for example, by intranasal administration. The antigen is typically administered with a suitable carrier and may optionally contain suitable immune system stimulants to increase the immune response. Suitable immune system stimulants include, but are not limited to, cytokines, growth factors, chemokines, mitogens and adjuvants. Suitable immune stimulants are well known to those skilled in the art and can be found, for example, in Plotkin and Orenstein, Vaccines, Third Ed., W.B. Saunders, 1999; Roitt et al., Immunology, Fifth Ed., Mosby, 1998; and Brostoff, et al., Clinical Immunology, Gower Medical Publishing, 1991. Examples of immune stimulants, include, but are not limited to, Alum (aluminum phosphate or aluminum hydroxide), Freund's adjuvant, calcium phosphate, beryllium hydroxide, saponins, polyanions, e.g. poly A:U, Quil A, inulin, lipopolysaccharide endotoxins, liposomes, lysolecithins, zymosan, propionibacteria, mycobacteria, and cytokines, such as, interleukin-1, interleukin-2, interleukin-4, interleukin-6, interleukin-12, interferon-a, interferon-g, and granulocyte-colony stimulating factor. Administration of the antigen can be accomplished by a single administration or by an initial administration followed by subsequent administrations (boosters). The boosters can be given at regular intervals and/or administered when high antibody titers are desired, for example, prior to harvesting antisera.

Once the antigen has been administered, the antibodies produced can be obtained from the blood serum. If the animal to which antigen is administered is a bird, for example a domestic chicken, antibodies can be obtained from the egg yolk using standard published methodologies (Svendsen et al., Lab. Anim. Sci., 45:89-93, 1995; Gassmann et al., FASEB J, 4:2528-2532, 1990). Such polyclonal antisera can be used as is or can be subjected to purification steps to further isolate the immunoglobulins contained in the antisera. Methods for the purification of immunoglobulins from sera are well known in the art and are further described in the examples herein.

The present invention also encompasses the use of neutralizing monoclonal antibodies. The production of monoclonal antibodies is routine in the art. Methods for the production of monoclonal antibodies to PRRSV are known and can be found, for example, in U.S. Pat. No. 5,677,429. Briefly, an animal is administered an antigen of interest as previously described. Once the animal has mounted an immune response, antibody-secreting cells are isolated, typically from spleen cells, but other sources of antibody secreting B cells such as blood can be used. Once obtained, the cells are immortalized commonly by fusion to a myeloma cell line or by viral transformation such as by Epstein-Barr virus transformation. The immortalized (hybridoma) cells are then selected, grown and tested for secretion of antibodies directed against the antigen of interest. Once suitable cells are identified, they are clonally expanded and the clonal cells are tested for antibody production. Once cells have been identified, they can be grown and antibody harvested from the culture medium. Alternatively hybridoma cells can be injected intraperitoneally into mice and the antibodies isolated from the ascites fluid produced. Methods for the purification of monoclonal antibodies from cell culture or ascites fluid are well known in the art and can be found in standard references such as Ausubel et al., unit 11. Monoclonal antibodies used in the practice of the present invention can be homologous, that is derived from antibody secreting cells from an animal of the same species as the animal to which they are administered or heterologous, that is derived from cells of a different species. In one embodiment, the monoclonal antibodies used are from a mouse monoclonal cell line. In another embodiment, the monoclonal antibodies used are derived from porcine antibody secreting B cells.

The present invention also encompasses the use of recombinant antibodies. One method for the production of recombinant antibodies is by the phage display method. Methods for the production and selection of antibodies using phage display are well known in the art and can be found, for example in Vaughan et al., Nature Biotech. 16:535-539, 1998; Watkins and Ouwehand, Vox Sanguinis 78:72-79, 2000; and the references cited therein.

Antibody production by phage display involves the generation of combinatorial libraries of immunoglobulin variable heavy chain (VH) and variable light chain (VL) sequences. These sequences are inserted into phage genes encoding coat proteins so that the VH and VL sequences are expressed (displayed) on the coat of filamentous bacteriophage. Phage expressing VH and VL regions of interest are selected by an affinity selection process commonly referred to as panning.

VH and VL sequences are generated by isolating mRNA from antibody secreting B-cells and amplifying the mRNA by reverse transcriptase-PCR (RT-PCR) using primers to conserved regions of the immunoglobulin gene. mRNA can be obtained from B-cells obtained directly from an animal, preferably an animal immunized with the antigen of interest, or from hybridoma cells producing antibodies against the antigen of interest. Once obtained, the VH and VL cDNA can be recombined by sequential cloning of VB and VL sequences into the same vector (Huse et al., Science, 246:1275-1281, 1989), by combinatorial infection using the loxCre site-specific recombination system of bacteriophage P1 (Waterhouse et al., Nuc. Acids Res., 21:2265-2266, 1993), or by PCR assembly (Clackson et al., Nature, 352:624-628, 1991; Marks et al., J Molec. Biol., 222:581-597, 1991). Alternatively, synthetic repertories of variable region sequences can be used as described, for example, in Griffiths et al. (EMBO J, 13:3245-3260, 1994).

Once a phage display library has been constructed, phage displaying reactive antibodies are selected by panning. Typically, purified antigen is attached to a solid substrate such as a plastic surface or an affinity chromatography column. The antigen may be attached to the surface directly or through an intermediary such as the streptavidin/biotin system. Phages to be selected are incubated with the antigen and non-binding phage washed away. A single round of selection can enrich for specific phage by 20 to 1,000 fold. Typically, several rounds of selection are carried out to increase specificity and affinity. Once phage displaying antibodies of the desired characteristics are identified, they are grown in a suitable bacterial host, the DNA encoding the antibody is isolated, and the DNA can then be sequenced. The antibody sequence can then be inserted into a suitable host cell for expression. Methods for the large scale production of antibodies from prokaryotic, lower eukaryotic and eukaryotic cells are well known in the art and can be found for example in Frenken et al., (Res. Immunol., 149:589-599, 1998) and the references cited therein.

Alternatively, chimeric antibodies can be used. Chimeric antibodies are those in which different regions of the immunoglobulin molecule are from different sources. Typically, chimeric antibodies comprise a mouse variable region and a constant region derived from the species to which the antibody is to be administered. Production of chimeric antibodies has become routine in the art and does not require any in depth structural knowledge of the antibody-antigen interaction (Watkins and Ouwehand, Vox Sanguinis, 78:72-79, 2000). Another form of chimeric antibody can be produced by the process known as “CDR grafting” (Jones et al., Nature, 321:522-525, 1986). CDRs are apical loops between the anti-parallel b-pleated sheets of a structure known as the immunoglobulin fold. The b-pleated sheets form a framework to correctly orientate the CDRs for interaction with the antigen. In CDR grafting, murine CDRs of a specific antibody are grafted onto an appropriate b-pleated sheet framework.

Additionally, antibodies can be obtained from transgenic animals and in particular transgenic mice (Bruggemann and Taussig, Curr. Opin. Biotechnol., 8:455-458, 1997). In this method, the endogenous mouse immunoglobulin genes are inactivated and replaced with unrearranged immunoglobulin sequences from the species of interest. Monoclonal antibodies of the species of interest are then produced from the transgenic mice using the methods described above.

Once obtained, the antibody containing serum, culture medium or ascites fluid can be administered without further processing or the immunoglobulins can be further isolated. Methods for the isolation of immunoglobulins is well known in the art and can be found in standard references such as Ausubel et al., unit 11. Any suitable method for the purification of immunoglobulins can be used. For example, immunoglobulins can be purified by affinity chromatography. Suitable affinity chromatography media include protein A chromatography columns, columns to which purified antigens are bound, and columns containing antibodies directed against immunoglobulins. Antibody purification can also be achieved using DEAE-Affi-Gel Blue chromatography columns. In one embodiment, immunoglobulins are isolated by ammonium sulfate fractionation followed by dialysis.

The unprocessed antibody containing fluid or the purified antibody fractions cam be further concentrated if desired. Any suitable method of concentration can be used. Exemplary methods of concentration include ultra filtration and lyophilization.

In the present method, animals are administered an effective amount of a composition containing anti-PRRSV antibodies. By an effective amount is meant an amount of anti-PRRSV antibodies that prevent intra uterine infection of fetuses, or which prevent/reduce clinical symptoms of PRRS or reduce titers of PRRSV or both. Any suitable method for the administration of the antibody containing composition can be used in the practice of the present invention. In one embodiment, the antibody containing composition is administered parenterally. Preferred methods of parenteral administration include subcutaneous, intravenous, intra-arterial, intramuscular and intraperitoneal administration.

The antibody containing compositions used in the present invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the composition may be in powder form, e.g. lyophilized, for reconstitution with a suitable vehicle, e.g., sterile pyrogen-free water, before use.

Antibody containing compositions for use in the present invention may comprise serum, concentrated serum or purified immunoglobulins, either alone or in combination with a pharmaceutically acceptable diluent, carrier and/or vehicle. Injectable antibody containing compositions, for example, sterile injectable aqueous or oleaginous suspensions, can be formulated according to the known art using suitable dispersing or wetting agents and suspending agents. Non-limiting examples of acceptable diluent, carriers and vehicles that may be employed are water, Ringer's solution, and isotonic sodium chloride solution. In addition, sterile, fixed oils may be employed as a vehicle or suspending medium. For this purpose, any bland fixed oil may be employed, including synthetic monon- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

### EXAMPLES

The following examples are intended to provide illustrations of the application of the present invention. The following examples are not intended to completely define or otherwise limit the scope of the invention.

### Example 1

### Production of Hyperimmune Sera

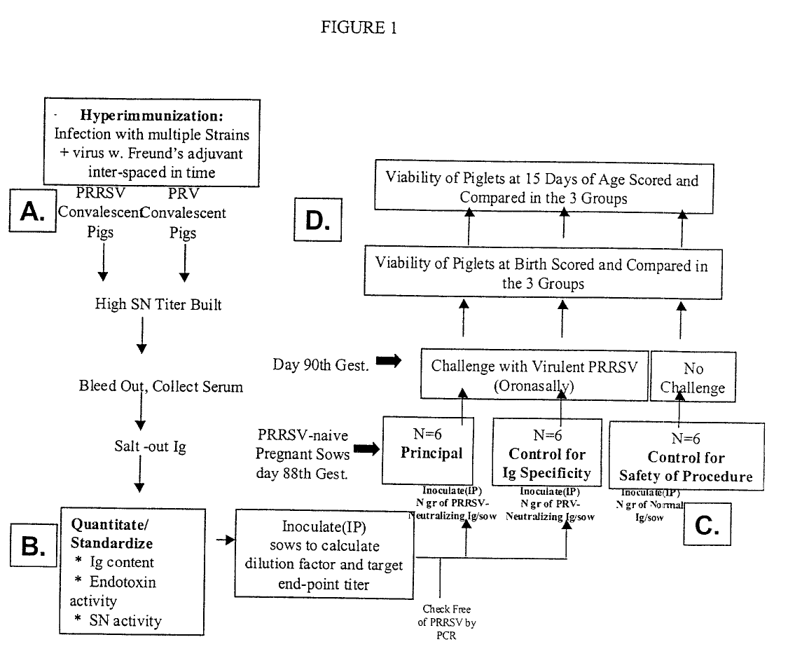

The basic plan followed is shown in FIG. 1. The virulent strain used for this experiment had been previously tested [“atypical PRRSV” 1-4-2 isolate from NADC, Ames, IA (Mengeling et al., Vet. Res., 59:1540-1544, 1998; Osorio, et al., Allen D. Lennan Swine Conf 25:176-182, 1998)]. The challenge with PRRSV took place via the oro-nasal route using this highly virulent PRRSV strain which ensures ˜100% neonatal mortality in PRRSV-free pregnant females, when inoculated at day 90 of gestation.

Block A (FIG. 1) shows the initial steps of the preparation of the master stock solution of PRRSV neutralizing antibodies. For the hyperimmunization, a total of thirteen PRRSV-free animals of approximately 300 lbs. of body weight were initially infected by oro-nasal route with the “atypical” PRRSV strain Iowa 1-4-2, which is hereby referred to as homologous strain for further challenge ( Block C/D, FIG. 1). At 5 weeks post infection (PI), the animals were super-infected with a mixture of 4 additional viable PRRSV strains, which included: 16244B (Allende, et al., J Gen. Virol., 80:307-315, 1999; Allende et al., Arch. Virol., 145:1149-1161, 2000), 2332 (ATTC Accession No.: VR-2332), NOBL (INGELVACâ PRRS MLV, Boehringer Ingelheim Animal Health, Ridgefield CT) and Schering (PrimePac PRRS MLV, Schering-Plough, Elk Horn, NE) vaccines. After 4 weeks following this secondary exposure to multiple PRRSV strains, each of the animals received a dose of 10⁵ TCID₅₀ (tissue culture infective dose₅₀) of the IA1-4-2 strain emulsified in 2 ml of Freund's complete adjuvant via intra-muscular route. At intervals of a month and half thereafter, the animals received a similar (10⁵ TCID₅₀) dose of homologous strain emulsified in 2 ml of Freund's incomplete adjuvant for a range of 3 to 6 applications. The titer of neutralizing antibodies in the peripheral blood of the immunized animals was monitored by a rapid neutralization assay of fluorescent foci on MARC 145 cells (FFN assay, conducted by Dr. Eric Nelson, Department of Veterinary Sciences, South Dakota State University) and was confirmed by a regular 4-day SN assay on Marc-i145 and porcine alveolar macrophages as well (Dr. Richard Hesse, Intervet Labs, Des Monies, Iowa). In all cases., there was a close correlation between the neutralization end-point titer when read on MARC 145 cells or on porcine alveolar macrophages. The SN endpoint titer against the homologous (challenge) PRRSV strain gradually progressed in each of the hyperimmunized animals. During a time period that ranged between 7 months (including 3 applications of virus + incomplete Freund's) and 14 months (including 6 applications of virus + incomplete Freund's), all of the 13 animals reached a final end-point titer that ranged between 1:32 to 1: 128, with most of the animals exhibiting an endpoint of at least 1:64. This point was considered to be the end of hyperimmunization process. The animals were exsanguinated and all of their serum individually collected. Serum immunoglobulins were precipitated and concentrated by ammonium sulfate treatment as described in Ausubel et al., unit 11.

A master stock solution of Pseudorabies Igs from PRRSV-free animals obtained from the same farm and with the same genetics as those previously used to hyperimmunize against PRRSV was prepared by vaccination of 8 animals of approximately 300 lbs. with a Pseudorabies virus (PRV) modified live virus vaccine (Syntrovet Marker Blue, Syntrovet, Lennexa, Kans.) followed by intranasal and conjunctival inoculation of wt PRV (Becker) strain. Two months post—wt PRV infection, all of the animals had reached a 1:32/1:128 range in their SN endpoints against PRV. The animals were likewise killed, bled out, and the PRV Ig salted out in a similar manner as previously described.

A third master solution of normal immunoglobulins collected from specific pathogen free (SPF) herds (PRRSV and PRV-free) was prepared following the same procedures. Block B (FIG. 1) indicates the phase of quantitation of immunoglobulins, (measured by an indirect ELISA anti-swine IgG (Bethyl Labs., Inc., Montgomery, Tex.). Likewise the level of endotoxin contamination was measured and equated in all of the three master immunoglobulin solutions to be ≦1 :64,000 (PYROTELLâ limulus amebocyte lysate test kit, Associates of Cape Cod, Falmouth, Mass.), while the level of interferon activity was found to be low and equivalent in all of the 3 master stock solutions. The interferon assay was conducted by VSV challenge in cell cultures using the method of Yousefi et al. (Am. J Clin. Pathol. 83:735-740, 1985). The end-point of PRRSV neutralizing activity attained for the corresponding master stock solution reached 1:256, while the stock with PRV neutralizing activity reached 1:512 by regular PRV SN assay. As indicated in block B (FIG. 1) both stock solutions were tested using at least two pregnant gilts of the same genetics and source as the rest of experimental animals The objective of this test was to measure the end-point SN titer attained after absorption into the circulation of the entire dose of immunoglobulin given intraperitoneally (body-dilution factor). In both cases, the end point was 1/16 at 48 hours after IP instillation.

### Example 2

### Passive Immunization

Blocks C & D of FIG. 1 describe the experimental design for the passive immunization experiment. Three homogeneous groups of 6 pregnant (87th day of gestation) gilts each were intraperitoneally instilled with 1.5 liters of Ig stock. One group (Principal Group) received PRRSV Igs (70 mg/ml of Igs and a PRRSV neutralizing end-point of 1:256), a second group (Control of Ab Specificity) received PRV Igs (70 mg/ml), and a third group (Control of the Safety of the IP Instillation Procedure) received normal Igs (60-70 mg/ml). The gilts received the IP instillation in standing position, with slight local anesthesia at the point of injection, using an atraumatic T-cannula. The time of instillation of the entire dose of Ig stock solution was 10-15 minutes/gilt. Intra-peritoneal instillation, at day 87 of gestation, of 1.5 liters of Ig stock solution consistently lead to the establishment of a detectable titer in peripheral blood of 1:16 (PRRSV SN) when samples were collected at day 90 of gestation (72 hrs after IP instillation). Likewise, the peripheral titers attained at day 90 of gestation upon instillation of 1.5 liters of PRV master stock Ig solution reached 1:32/1:64 (PRV SN)

### Example 3

### PRRSV Challenge

At 3 days after IP instillation, two of the groups (Principal and Ab Specificity control, Block C, FIG. 1) were challenged oronasally with 10^{5.4} TCID₅₀ of Iowa 1-4-2 (second passage in MARC 145). The third group (Control of Safety of the Instillation Procedure) was not challenged. The three groups were maintained in isolation from each other during the entire experiment.

The offspring viability scores are shown in Table 1. Clinically there was no significant alterations in the Principal (PRRSV Abs, challenged) and the Safety of Procedure Control (Normal Igs, unchallenged) groups. The animals maintained their normal appetite and alertness during the remaining gestation time, and did not show any clinical symptoms of PRRS. On the other hand, the six animals of the Ab Specificity Control Group ( PRV-Abs, challenged) presented significant clinical alterations starting at 24 hrs after PRRSV challenge. The animals were clearly lethargic, lacked appetite and presented mild fever and rough appearance for the rest of the gestation period. In this group, three of the farrowings were advanced by 7, 6 and 5 days of the anticipated due date, respectively (see Table 1 for viability scores).

The scores for offspring viability at birth and at weaning following challenge (Table 1) clearly show a significant difference as determined by analysis of variance (Proc GLM SAS program, SAS Institute, Cary, N.C.) between those groups that received either PRRSV-neutralizing antibodies (Principal Group), or normal Ig and no challenge (Procedure Control Group), and the Ab Specificity Control Group which received an equivalent amount of an unrelated (PRV) neutralizing antibody and PRRSV challenge. The Ab Specificity Control Group exhibited unequivocal signs of reproductive failure which are characteristic of the highly virulent strain that was used as a challenge. The 6 gilts of this group (specially those that presented advanced farrowings at 5-7 days prior to estimated due date) delivered most of their litters dead (decomposed +stillborns) with just a few piglets surviving for a few (1-3) days. In addition to the significant mortality at birth, the survival of piglets born alive in this group was dramatically lower (Table 1). The presence of PRRSV was confirmed in all of the aborted litters by immunohistochemistry in thymus and other lymphoid tissue, as well as by viral isolation or PCR identification in the thoracic fluid of the fetuses. The 3 piglets from this group that were still alive at weaning (two piglets from sow No. 229 and one from sow No. 230, Table 1) were necropsied at the time of weaning. These piglets showed evident pneumonitis, with their lymph nodes significantly enlarged and hemorrhagic, as is characteristic of PRRS in young pigs. The PRRSV was isolated from several tissues of these animals and detected by immunohistochemistry in their lymphoid tissues.

The principal group (PRRSV antibodies, challenge) did not exhibit significant mortality, and the size and appearance of the litters exhibited no significant differences with the non challenged Procedure Control Group by analysis of variance (Proc GLM SAS). The few sporadic natal or perinatal deaths observed in these two groups were within the normal range and were due to normal causes (i.e., trauma caused by the darn, supernumerary litters, etc). No evidence of PRRSV infection was found in any of the 4 piglets born dead in the Principal Group or in any of the 3 piglets born dead in the Procedure Control Group (Table 1). The survival of the piglets born alive reached 100% in the Principal Group and 95% in the Procedure Control Group.

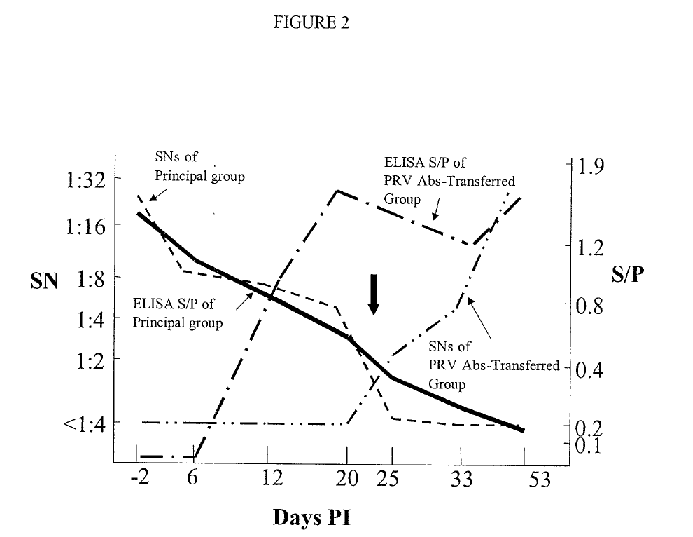

FIG. 2 shows the PRRSV serologic profiles of the Principal and Control of Ab Specificity Groups composed of the mean SN and ELISA(S/P) titers. The transferred PRRSV antibodies follow a continuous decline starting from the titers reached immediately after transfer. The SN activity decayed to undetectable levels in the gilts at around the time of farrowing, with a concomitant increase in colostrum (i.e. with SN titers reaching up to 1:128 in colostrum) and milk and subsequent transfer to the piglets (data not shown). The S/P of ELISAs, while decaying more slowly, reached near negligible titers at the end of the experiment (weaning of the offspring at day 15 of age)(FIG. 2). The decline of passively acquired PRV antibodies in the Control of Ab Specificity Group, which occurred at a kinetics equivalent to the decay to the PRRSV antibodies, is not shown. The serologic profile (SN and ELISA) of the group of animals that received unrelated (PRV) antibodies (FIG. 2) corresponds to a typical primary response upon exposure to PRRSV, with the characteristic precedence of the ELISA response over a more delayed SN response.

Virological examination also indicated significant differences among both groups challenge with PRRSV. Viremia titers ranging between 4 and 5 log units were detected in all gilts (n=6) of the PRV-Abs treated group during the first two weeks post-infection (PI), but in none of the gilts of the principal (PRRSV-Abs treated) group. These results were confirmed by PCR, which was positive for PRRSV for sera from all members of the PRV-Abs treated group but in none of the members of the Principal Group. Likewise, individual virological and PCR examination of the tissues of gilts of the PRRSV-Abs treated group (Principal Group) failed to indicate the presence of PRRSV. Importantly, the serology of all of the healthy litters farrowed by the Principal group indicated a high titer of neutralizing antibodies transferred from the dam at farrowing time followed by a declined to complete absence of antibodies (by SN and by ELISA) by day 45 of age (data not shown). The absence of a rise in antibodies that could suggest continuous infection with live PRRSV upon disappearance of maternal antibodies emphasizes that the animal were born free of PRRSV infection.

TABLE 1
Offspring Viability Scores at Birth and at 15 days of age (weaning time)

| | Abs used | | | Viability | | Viability |
|---|---|---|---|---|---|---|
| | for passive | | | at Birth | | at 15 |
| GROUP | transfer | Sow # | | DEAD | LIVE | Days of age |
| | | 229 | | 9 | 2 | 2 |
| | | | 230 | 11 | 1 | 1 |
| Control | PRV | | 231 | 7 | 3 | 0 |
| of Ab | | | 232 | 10 | 4 | 0 |
| Specificity | | | 233 | 9 | 2 | 0 |
| | | | 241 | 13 | 1 | 0 |
| | | 234 | | 0 | 14 | 14 |
| | | | 235 | 1 | 13 | 13 |
| Principal | | | 236 | 1 | 10 | 10 |
| | PRRSV | | 237 | 1 | 13 | 13 |
| | | | 238 | 1 | 11 | 11 |
| | | | 239 | 0 | 5 | 5 |
| | | 244 | | 1 | 10 | 10 |
| | | | 248 | 0 | 10 | 10 |
| Control | Normal | | 249 | 2 | 11 | 9 |
| of Safety | Igs | | 242 | 0 | 16 | 14 |
| Of IP | | | 243 | 0 | 12 | 12 |
| Instillation | | | | | | |
| Procedure | | | | | | |

### Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for the prevention of intra uterine infection of fetuses with Porcine Reproductive and Respiratory Syndrome virus (PRRSV) comprising, administering to a pregnant female a composition containing an effective amount of anti-PRRSV antibodies.

2. The method of claim 1, wherein said composition is administered during the third trimester of pregnancy.

3. The method of claim 1, wherein said composition comprises polyclonal anti-PRRSV antibodies.

4. The method of claim 3, wherein said composition comprises hyperimmune serum.

5. The method of claim 1, wherein said composition comprises at least one anti-PRRSV monoclonal antibody.

6. The method of claim 1, wherein said composition comprises at least one recombinant anti-PRRSV antibody.

7. The method of claim 1, wherein said composition is administered parenterally.

8. The method of claim 7, wherein said parenteral administration is intraperitoneal, intravenous, subcutaneous, intramuscular, or intra-arterial.

9. The method of claim 1, wherein said composition is administered prior to exposure of said pregnant female to said Porcine Reproductive and Respiratory Syndrome Virus.

10. The method of claim 1, wherein said composition is administered after exposure of said pregnant female to said Porcine Reproductive and Respiratory Syndrome Virus.

11. The method of claim 1 wherein said effective dose is about 100 g of immunoglobulin with a PRRSV serum neutralizing end point of 1:256.

12. The method of claim 1, wherein said effective dose results in a PRRSV serum neutralizing antibody titer of 1:16 by about 3 days post administration.

13. The method of claim 1, wherein said antibody containing composition is administered in conjunction with a vaccine.

14. The method of claim 13, wherein said vaccine is directed against an organism or disease selected from the group consisting of PRRSV, hog cholera, transmissible gastroenteritis, swine influenza, porcine parvovirus, swine enzootic pneumonia, pseudorabies, encephalomyocarditis, Japanese encephalitis, rotavirus, leptospirosis, erysipelas, brucellosis, enterovirus, and any combination thereof.

15. A method for the prevention of intra uterine infection of fetuses with Porcine Reproductive and Respiratory Syndrome Virus (PRRSV) comprising parenterally administering to a pregnant female during the last trimester of gestation an effective amount of a composition containing anti PRRSV antibodies.

16. The method of claim 15, wherein said effective dose is about 100 g of immunoglobulin with a PRRSV serum neutralizing end point of 1:256.

17. The method of claim 15, wherein said effective dose results in a PRRSV serum neutralizing antibody titer of 1:16 by about 3 days post administration.

18. The method of claim 15, wherein said anti PRRSV antibody containing composition is administered in conjunction with a vaccine.

19. The method of claim 18, wherein said vaccine is directed against an organism or disease selected from the group consisting of PRRSV hog cholera, transmissible gastroenteritis, swine influenza, porcine parvovirus, swine enzootic pneumonia, pseudorabies, encephalomyocarditis, Japanese encephalitis, rotavirus, leptospirosis, erysipelas, brucellosis, enterovirus, and any combination thereof.

20. A method for the prophylaxis or treatment of Porcine Reproductive and Respiratory Syndrome (PRRS) in swine comprising administering to a pig a composition comprising an effective amount of anti PRRS virus antibodies.

21. The method of claim 20, wherein said pig is a pregnant female.

22. The method of claim 21, wherein said administration is during the last trimester of pregnancy.

23. The method of claim 20, wherein said pig is a neonatal pig.

24. The method of claim 20, wherein said pig is a weanling pig.

25. The method of claim 20, wherein said composition comprises polyclonal anti-PRRSV antibodies.

26. The method of claim 25, wherein said composition comprises hyperimmune serum.

27. The method of claim 20, wherein said composition comprises at least one anti-PRRSV monoclonal antibody.

28. The method of claim 20, wherein said composition comprises at least one recombinant anti-PRRSV antibody.

29. The method of claim 20, wherein said composition is administered parenterally.

30. The method of claim 29, wherein said parenteral administration is intraperitoneal, intravenous, subcutaneous, intramuscular, or intra-arterial.

31. The method of claim 20, wherein said effective dose is about 100 g of immunoglobulin with a PRRSV serum neutralizing end point of 1:256.

32. The method of claim 20, wherein said effective dose results in a PRRSV erum neutralizing antibody titer of 1:16 by about 3 days post administration.

33. The method of claim 20, wherein said anti-PRRSV antibody containing composition is administered in conjunction with a vaccine.

34. The method of claim 33, wherein said vaccine is directed against an organism or disease selected from the group consisting of PRRSV, hog cholera, transmissible gastroenteritis, swine influenza, porcine parvovirus, swine enzootic pneumonia, pseudorabies, encephalomyocarditis, Japanese encephalitis, rotavirus, leptospirosis, erysipelas, brucellosis, enterovirus, and any combination thereof.

35. A composition comprising an effective amount of anti PRRS virus antibodies.

36. The composition of claim 35 wherein the anti PRRS virus antibodies are polyclonal antibodies.

37. The composition of claim 35 wherein the anti PRRS virus antibodies are monoclonal antibodies.

38. The composition of claim 35 wherein the anti PRRS virus antibodies are recombinant antibodies.

39. The composition of claim 35 wherein the composition further comprises a vaccine.
